(19) United States
(12) Patent Application Publication
(10) Pub. No.: US 2008/0132471 A1
(43) Pub. Date: Jun. 5, 2008

(54) BISPHOSPHONATE INHALANT FORMULATIONS AND METHODS FOR USING THE SAME

(75) Inventors: Toru Hibi, San Jose, CA (US); Akira Yamamoto, San Jose, CA (US); Maria Nakatani, San Jose, CA (US); Hidemasa Katsumi, San Jose, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: TEIKOKU PHARMA USA, INC.

(21) Appl. No.: 11/935,764

(22) Filed: Nov. 6, 2007

Related U.S. Application Data
(60) Provisional application No. 60/866,787, filed on Nov. 21, 2006.

(51) Int. Cl. A61K 31/66; A61K 31/675; A61P 9/00

(52) U.S. Cl. 514/89; 514/108; 514/94

(57) ABSTRACT

The present invention provides for methods of administering by a pulmonary route an effective amount of a bisphosphonate active agent to a subject. Aspects of the invention including administering the active agent to the subject in conjunction with one or more mucosal membrane protecting agents, where the protecting agent may include one or more of a protecting enzyme and/or a protecting amino acid and/or a protecting peptide. Also provided are inhalant compositions for use in practicing methods according to embodiments of the invention. Methods and compositions according to embodiments of the invention find use in a variety of different applications, including but not limited to, the treatment of bone adsorption disease conditions.

### CROSS-REFERENCE TO RELATED APPLICATIONS

Pursuant to 35 U.S.C. § 119 (e), this application claims priority to the filing date of U.S. Provisional Patent Application Ser. No. 60/866,787 filed Nov. 21, 2006; the disclosure of which is herein incorporated by reference.

### INTRODUCTION

Bisphosphonates and their pharmacologically acceptable salts find use in a variety of different applications. For example, bisphosphonates have been employed as bone absorption inhibitors in treating patients suffering from osteoporosis, Paget's disease and cancer.

In the past, bisphosphonates have been administrated orally and intravenously. However, there are disadvantages associated with the oral and intravenous administration of bisphosphonates. For example, the bioavailability of a bisphosphonate following oral administration can be very low. Furthermore, bisphosphonates can be irritating to the gastrointestinal tract. In addition, patient compliance can be problematic as patients are typically prevented from lying down following oral administration.

Intravenous administration of bisphosphonates, while overcoming some of the disadvantages of oral administration, is not entirely satisfactory. For example, because rapid intravenous administration of bisphosphonates may cause renal complications, intravenous bisphosphonate administration generally takes a long period of time.

Because of the above disadvantages of oral and intravenous bisphosphonate administration, inhalation administration of bisphosphonates has been proposed. See e.g., U.S. Pat. No. 6,743,414. However, inhalation administration of bisphosphonates can be damaging to the pulmonary mucosal tissue.

### SUMMARY

The present invention provides for methods of administering by a pulmonary route an effective amount of a bisphosphonate active agent to a subject. Aspects of the invention including administering the active agent to the subject in conjunction with one or more mucosal membrane protecting agent, such as a protecting enzyme and/or a protecting amino acid and/or a protecting peptide. Also provided are inhalant compositions for use in practicing methods according to embodiments of the invention. Methods and compositions according to embodiments of the invention find use in a variety of different applications, including but not limited to, the treatment of bone adsorption disease conditions.

### BRIEF DESCRIPTION OF THE FIGURES

FIG. 1 provides a graph of the observed plasma concentration profile of alendronate after its administration to rats by intravenous or intrapulmonary administration, as reported in the Experimental Section, below.

FIG. 2 provides a graph of the observed plasma concentration profile of pamidronate after its administration to rats by intravenous or intrapulmonary administration, as reported in the Experimental Section, below.

FIG. 3 provides a graph of the observed LDH activity in bronchoalveolar lavage fluid (BALF) at 4 hr after intrapulmonary administration of alendronate with SOD, cysteine, taurine, glutathione in rats, as reported in the Experimental Section, below.

FIG. 4 provides a graph of the observed LDH activity in bronchoalveolar lavage fluid (BALF) at 4 hr after intrapulmonary administration of pamidronate with SOD and cysteine in rats, as reported in the Experimental Section, below.

### DEFINITIONS

When describing the compounds, pharmaceutical compositions containing such compounds and methods of using such compounds and compositions, the following terms have the following meanings unless otherwise indicated. It should also be understood that any of the moieties defined forth below may be substituted with a variety of substituents, and that the respective definitions are intended to include such substituted moieties within their scope.

“Alkyl” refers to monovalent saturated aliphatic hydrocarbyl groups particularly having up to 10 carbon atoms, or up to 9 carbon atoms, up to 8 carbon atoms, or up to 3 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “alkyl” also includes “cycloalkyls” as defined herein.

“Cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like.

“Heterocycloalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of such heterocyclic non-aromatic rings include, but are not limited to, aziridinyl, azetidinyl, piperazinyl, and piperidinyl.

“Heteroaryl” refers to a stable heterocyclic aromatic ring and fused rings containing one or more heteroatoms independently selected from N, O and S. A fused heterocyclic ring system may include carbocyclic rings and need only include one heterocyclic ring. Examples of such heterocyclic aromatic rings include, but are not limited to, pyridine, pyrimidine, and pyrazinyl.

“Aryl” refers to a monovalent aromatic hydrocarbon group derived by the removal of one hydrogen atom from a single carbon atom of a parent aromatic ring system. Typical aryl groups include, but are not limited to, groups derived from benzene, ethylbenzene, mesitylene, toluene, xylene, aniline, chlorobenzene, nitrobenzene, and the like.

“Aralkyl” or “arylalkyl” refers to an alkyl group, as defined above, substituted with one or more aryl groups, as defined above.

“Halogen” refers to fluoro, chloro, bromo and iodo. In some embodiments, the halogen is fluoro or chloro.

“Substituted” refers to a group in which one or more hydrogen atoms are each independently replaced with the same or different substituent(s). “Substituted” groups particularly refer to groups having 1 or more substituents, for instance from 1 to 5 substituents, and particularly from 1 to 3 substituents, selected from the group consisting of amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryl, substituted thioaryl, thioketo, thiol, alkyl-S(O)—, aryl-S(O)—, alkyl-S(O)₂— and aryl-S(O)₂—.

### DETAILED DESCRIPTION

The present invention provides for methods of administering by a pulmonary route an effective amount of a bisphosphonate active agent to a subject. Aspects of the invention including administering the active agent to the subject in conjunction with a mucosal membrane protecting agent, such as a protecting enzyme and/or a protecting amino acid and/or a protecting peptide. Also provided are inhalant compositions for use in practicing methods according to embodiments of the invention. Methods and compositions according to embodiments of the invention find use in a variety of different applications, including but not limited to, the treatment of bone adsorption disease conditions.

Before the present invention is described in greater detail, it is to be understood that this invention is not limited to the particular embodiments described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

In further describing the subject invention, the subject methods are described first in greater detail, followed by a review of the various compositions, e.g., formulations and kits, that may find use in the subject methods, as well as a discussion of various representative applications in which the subject methods and compositions find use.

### Methods

Aspects of the invention include methods of administering a bisphosphonate active agent to a subject. The subject may be in need thereof, e.g., for the treatment of a disease or condition treatable by a bisphosphonate active agent (as described in greater detail below). Aspects of the subject methods include administering a bisphosphonate active agent to a subject in combination with a mucosal membrane protecting agent. In some embodiments, the mucosal membrane protecting agent is a protecting enzyme. In some embodiments, the mucosal membrane protecting agent is a protecting amino acid. In some embodiments, the mucosal membrane protecting agent is a protecting peptide. In some embodiments, the active agent is administered in combination with two or three of a protecting enzyme, a protecting amino acid and a protecting peptide.

By “in combination with” is meant that an amount of the mucosal membrane protecting agent(s) is administered anywhere from simultaneously to up to 5 hours or more, e.g., 10 hours, 15 hours, 20 hours or more, prior to or after the bisphosphonate active agent. In certain embodiments, the bisphosphonate active agent and mucosal membrane protecting agent(s) are administered sequentially, e.g., where the bisphosphonate active agent is administered before or after the mucosal membrane protecting agent(s). In yet other embodiments, the bisphosphonate active agent and mucosal membrane protecting agent(s) are administered simultaneously to the subject, e.g., where the bisphosphonate active agent and mucosal membrane protecting agent(s) are administered to the subject at the same time as two separate formulations, or optionally as three separate formulations, or are combined into a single formulation that is administered to the subject. Regardless of whether the bisphosphonate active agent and mucosal membrane protecting agent(s) are administered sequentially or simultaneously, as illustrated above, the agents are considered to be administered together or in combination (i.e., in conjunction) for purposes of the present invention. Routes of administration of the two, or optionally three, agents may vary, where routes of administration of interest include, but are not limited to, those described in greater detail below.

### Bisphosphonate Active Agent

In the subject methods, a bisphosphonate active agent is administered to subject in combination with a mucosal membrane protecting agent(s). Bisphosphonate active agents of interest include bisphosphonate compounds that are capable of inhibiting the resorption of bone. Bisphosphonate compounds are also known as diphosphonates or bisphosphonic acid. The bisphosphonate active agent may have a high affinity to bone tissue. In some embodiments, the bisphosphonate active agent metabolizes in a cell into compounds that compete with adenosine triphosphate (ATP) in the cellular energy metabolism. In some embodiments, the bisphosphonate active agent binds the farynesyl disphosphate synthase (FPPS) enzyme and inhibits the enzymatic activity of FPPS. FPPS is an enzyme involved in the 3-hydroxy-3-methylglutaryl-CoA (HMG-CoA) reductase pathway (or mevalonate pathway). Bisphosphonate active agents useful in the subject compositions include, but are not limited to those compounds described in U.S. Pat. Nos. 4,621,077; 5,183,815; 5,358,941; 5,462,932; 5,661,174; 5,681,590; 5,994,329; 6,015,801; 6,090,410; 6,225,294; 6,414,006; 6,482,411; and 6,743,414; the disclosures of which are herein incorporated by reference.

Whether or not a given bisphosphonate active agent is suitable for use according to the present invention can be readily determined using assays employed in the experimental section, below. In certain embodiments, a bisphosphonate active agent is suitable for use in the subject methods if it exhibits desired activity as determined using the in situ trans-pulmonary absorption test described in the experimental section, below.

In certain embodiments, the bisphosphonate active agent of interest is a compound of formula (I):

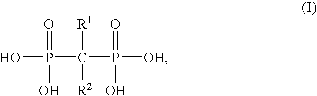

or the pharmaceutically acceptable salts, solvates, hydrates, and prodrug forms thereof, and stereoisomers thereof;

wherein:

R¹ is selected from the group consisting of hydrogen, hydroxy, and halogen; and

R² is selected from the group consisting of halogen, a linear or branched substituted or unsubstituted C₁-C₁₀ alkyl, a linear or branched substituted or unsubstituted C₁-C₁₀ cycloalkyl, a linear or branched substituted or unsubstituted C₁-C₁₀ aryl, a linear or branched substituted or unsubstituted C₁-C₁₀ aralkyl, a substituted or unsubstituted C₁-C₁₀ heterocycloalkyl, or a substituted or unsubstituted C₁-C₁₀ heteroaryl, wherein the each carbon atom of R² may be optionally replaced with a nitrogen or sulfur atom and R² has no more than 3 nitrogen or sulfur atoms in total.

In certain embodiments, R² is selected from the group consisting of halogen, a linear or branched substituted or unsubstituted C₁-C₈ alkyl, a linear or branched substituted or unsubstituted C₁-C₈ cycloalkyl, a linear or branched substituted or unsubstituted C₁-C₉ aryl, or a linear or branched substituted or unsubstituted C₁-C₈ aralkyl, wherein the each carbon atom of R² may be optionally replaced with a nitrogen or sulfur atom and R² has no more than 2 nitrogen or sulfur atoms in total, wherein R² has no more than 8 carbon atoms.

In certain embodiments, R² is a linear or branched C₁-C₈ alkyl, wherein the each carbon atom of R² may be optionally replaced with a nitrogen atom and the total number of nitrogen is R² is not more than 1, wherein the C₁-C₈ alkyl may be optionally substituted with an amino group.

In some embodiments, R¹ is hydroxy or fluorine and R² is fluorine or a linear or branched C₁-C₅ alkyl, which may optionally be substituted by a substituent such as amino groups and/or fluorine atoms, and their salts with alkali metals, organic bases and basic amino acids.

In some embodiments, R² is:

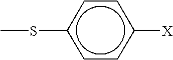

wherein X is a halogen.

In some embodiments, R² is:

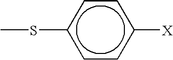

wherein X is a halogen; and R¹ is hydrogen.

In some embodiments, R² is —CH₃, —CH₂—CH₂—NH₂, —(CH₂)₅—NH₂,

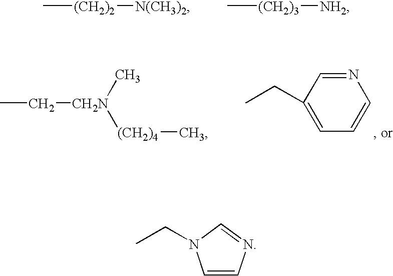

Specific bisphosphonate active agents of interest are shown in Table 1 (wherein the compound is of formula (I)):

TABLE 1

| Bisphosphonate | | |
|---|---|---|
| active agent | R¹ side chain | R² side chain |
| Etidronate | —OH | —CH₃ |
| Clodronate | —Cl | —Cl |
| Tiludronate | —H | 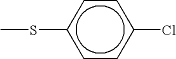 |
| Pamidronate | —OH | —CH₂—CH₂—NH₂ |
| Neridronate | —OH | —(CH₂)₅—NH₂ |
| Olpadronate | —OH | —(CH₂)₂—N(CH₃)₂ |
| Alendronate | —OH | —(CH₂)₃—NH₂ |
| Ibandronate | —OH | 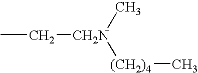 |
| Risedronate | —OH | 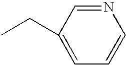 |
| Zoledronate | —OH | 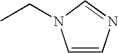 |

Specific bisphosphonates of interest include, but are not limited to: (4-amino-1-hydroxybutylidene)-bis-phosphonate or 4-amino-1-hydroxybutane-1,1-biphosphonic acid (alendronate); (Dichloromethylene)-bis-phosphonate (clodronate); (1-Hydroxyethylidene)-bis-phosphonate (etidronate); [1-Hydroxy-3-(methylpentylamino)propylidene]bis-phosphonate (ibandronate); [(Cycloheptylamino)-methylene]bis-phosphonate (incadronate); [1-Hydroxy-2-imidazo-(1,2-a)pyridine-3-ylethylidene]bis-phosphonate (minodronate); (6-amino-1-hydroxyhexylidene)bis-phosphonate (neridronate); [3-(Dimethylamino)-hydroxy-propylidene]bis-phosphonate (olpadronate); (3-Amino-1-hydroxypropylidene)bis-phosphonate (pamidronate); [1-Hydroxy-2-(3-pyridinyl)-ethylidine]bis-phosphonate (risedronate); [[4-Chlorophenyl)thio]-methylene]bis-phosphonate (tiludronate); [1-Hydroxy-2-(1H-imidazole-1-yl)ethylidene]bis-phosphonate (zoledronate); [(Cycloheptylamino)-methylene]bis-phosphonate (incadronate); [1-Hydroxy-2-imidazo-(1,2-a)pyridine-3-ylethylidene]bis-phosphonate (minodronate); 5-amino-1-hydroxypentan-1,1-biphosphonic acid; 4-amino-1-hydroxybutan-1,1-biphosphonic acid; difluoro-methanbiphosphonic acid; and pharmacologically acceptable salts thereof.

Pharmacologically acceptable salts include, are not limited to, salts of alkali metal (e.g., sodium and potassium), salts of alkali earth metals (e.g., calcium), salts of inorganic acids (e.g., HCl), and salts of organic acids (e.g., citric acids and amino acids, such as lysine). In one embodiment, the bisphosphonate active agent is a salt of sodium. When bisphosphonate active agent is alendronate, the monosodium salt trihydrate form of alendronate is employed in certain embodiments. In certain embodiments, the bisphosphonate active agent is in its anhydrous form.

### Mucosal Membrane Protecting Agent

By “mucosal membrane protecting agent” is meant an agent that reduces unwanted irritation caused by the bisphosphonate active agent when the bisphosphonate active agent is administered to the subject by a pulmonary route. As such, a mucosal membrane protecting agent is one that reduces bisphosphonate induced pulmonary irritation. Mucosal membrane protecting agents of interest are those agents that reduce bisphosphonate induced pulmonary irritation by about 2 to 10-fold or more, such as by about 50-fold or more, and including by about 100-fold or more, as determined using the in situ trans-pulmonary absorption test and the pulmonary inflammation test described in the experimental section, below.

Mucosal membrane protecting agents of interest include, but are not limited to: protecting enzymes, protecting amino acids and protecting peptides. In certain embodiments, a single mucosal membrane protecting agent is employed. In yet other embodiments, two or more different mucosal membrane protecting agents are employed, e.g.: a protecting enzyme and a protecting amino acid; a protecting enzyme and a protecting peptide; a protecting amino acid and a protecting peptide; two different protecting enzymes; two different protecting amino acids; two or more different protecting peptides; a protecting enzyme, protecting amino acid and a protecting peptide; etc.

Protecting enzymes of interest include enzymes capable of catalyzing the dismutation of superoxide into oxygen and hydrogen peroxide, e.g., as determined using the assay described in: Peskin et al. Clinica Chimica Acta 293:157-166, 2000.

Exemplary enzymes of interest include, but are not limited to: superoxide dismutase (SOD), glutathione-S-transferase, glutathione reductase, catalase, enzymatically active portions or variants thereof. Such enzymes are disclosed in U.S. Patent Application Pub. No. 2006/0165672. Suitable SODs include human SOD and bovine SOD. In some embodiments, the enzyme is a recombinant enzyme. Active portions of the enzyme are polypeptides that lack the full length amino acid sequence of an enzyme and retain at least a substantial part of the enzymatic activity of the enzyme. Active variants of the enzyme are polypeptides that contain insertion, deletion or substitution mutations of the amino acid sequence of an enzyme and retain at least a substantial part of the enzymatic activity of the enzyme.

A “substantial part of an enzymatic activity” is at least 50%, at least 70%, at least 80%, or at least 90% of the enzymatic activity of the full length enzyme.

“Recombinant” has the usual meaning in the art, and refers to an enzyme synthesized, expressed, or otherwise manipulated in vitro, methods of using recombinant polynucleotides or vectors (or a non-naturally occurring polynucleotide or vector) encoding an enzyme to produce the enzyme in cells or other biological systems (or a non-naturally occurring system), or an enzyme produced by such a method.

“Variant” refers to an enzyme having an amino acid sequence of a naturally occurring enzyme wherein the amino acid sequence of the variant enzyme is modified. Such variant enzymes necessarily have less than 100% sequence identity or similarity with the amino acid sequence of a naturally occurring enzyme, and having at least 75% amino acid sequence identity or similarity, or at least 80%, or at least 85%, or at least 90%, or at least 95% sequence identity or similarity with the amino acid sequence of a naturally occurring enzyme. Such modified amino acid sequences comprise the insertion, deletion and/or substitution of one or more amino acids of the original amino acid sequence of the naturally occurring enzyme.

Protecting amino acids of interest include, but are not limited to: taurine and cysteine, as well as pharmaceutically acceptable salts, solvates, and derivatives thereof.

Protecting peptides of interest include, but are not limited to: glutathione, as well as pharmaceutically acceptable salts, solvates, and derivatives thereof.

As indicated above, an effective amount of mucosal membrane protecting agent(s) is employed in the subject methods. In certain embodiments, the amount of mucosal membrane protecting agent employed is not more than about the amount of the bisphosphonate active agent employed. In other embodiments, the effective amount is the same as the amount of the active agent, and in certain embodiments the effective amount is an amount that is more than the amount of the bisphosphonate active agent. Effective amounts can readily be determined empirically using the data provided in the experimental section, below.

In some embodiments of the invention, the bisphosphonate active agent is alendronate, and the mucosal membrane protecting agent includes superoxide dismutase, taurine, cysteine and glutathione.

In some embodiments of the invention, the bisphosphonate active agent is pamidronate, and the mucosal membrane protecting agent includes superoxide dismutase, taurine, cysteine and glutathione.

### Formulations and Administration

Also provided are pharmaceutical compositions containing the bisphosphonate active agent and/or mucosal membrane protecting agent(s) employed in the subject methods. In certain embodiments, the bisphosphonate active agent and/or mucosal membrane protecting agent(s), e.g., in the form of a pharmaceutically acceptable salt, are formulated for pulmonary administration to a subject. In certain embodiments, e.g., where the compounds are administered as separate formulations (such as in those embodiments where they are administered sequentially), separate or distinct pharmaceutical compositions—each containing a different active agent, are provided. In some embodiments, a single formulation that includes both of the bisphosphonate active agent and mucosal membrane protecting agent(s) (i.e., one composition that includes both active agents) is provided.

By way of illustration, the bisphosphonate active agent and/or mucosal membrane protecting agent(s) can be admixed with conventional pharmaceutically acceptable carriers and excipients (i.e., vehicles) and used in forms suitable for pulmonary administration. Such suitable forms include aqueous solutions, suspensions, and the like. Such pharmaceutical compositions contain, in certain embodiments, from about 0.1 to about 90% by weight of the active compound, such as from about 1 to about 30% by weight of the active compound.

A liquid composition may be present as a suspension or solution of the compound or pharmaceutically acceptable salt in a suitable liquid carrier(s), for example, glycerine, sorbitol, non-aqueous solvent such as polyethylene glycol, oils or water, with a suspending agent, preservative, surfactant, wetting agent, flavoring or coloring agent. Alternatively, a liquid formulation can be prepared from a reconstitutable powder.

In certain embodiments of interest, the bisphosphonate active agent and the mucosal membrane protecting agent(s) are administered as a single pharmaceutical formulation, that, in addition to including an effective amount of each of the agents, includes other suitable compounds and carriers, and also may be used in combination with other active agents. The present invention, therefore, also includes pharmaceutical compositions comprising pharmaceutically acceptable excipients. The pharmaceutically acceptable excipients include, for example, any suitable vehicles, adjuvants, carriers or diluents, and are readily available to the public. The pharmaceutical compositions of the present invention may further contain other active agents as are well known in the art.

One skilled in the art will appreciate that a variety of suitable methods of administering a formulation of the present invention to a subject, are available, and, although more than one route can be used to administer a particular formulation, a particular route can provide a more immediate and more effective reaction than another route. Pharmaceutically acceptable excipients may be employed as desired. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting.

The subject formulations of the present invention can be made into aerosol formulations to be administered via inhalation. These aerosol formulations (i.e., inhalant formulations) can be placed into pressurized acceptable propellants, such as dichlorodifluoromethane, propane, nitrogen, and the like. They may also be formulated as pharmaceuticals for non-pressured preparations, such as for use in a nebulizer or an atomizer.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Those of skill in the art will readily appreciate that dose levels can vary as a function of the specific compound, the nature of the delivery vehicle, and the like. Suitable dosages for a given compound are readily determinable by those of skill in the art by a variety of means.

The dose administered to an animal, particularly a human, in the context of the present invention should be sufficient to effect a prophylactic or therapeutic response in the animal over a reasonable time frame. One skilled in the art will recognize that dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound. Suitable doses and dosage regimens can be determined by comparisons to bone adsorption inhibiting agents that are known to reduce bone loss due to bone adsorption.

Optionally, the pharmaceutical composition may contain other pharmaceutically acceptable components, such a buffers, surfactants, viscosity modifying agents, preservatives and the like. Each of these components is well-known in the art. See, e.g., U.S. Pat. No. 5,985,310, the disclosure of which is herein incorporated by reference. Other components suitable for use in the formulations of the present invention can be found in Remington's Pharmaceutical Sciences, Mace Publishing Company, Philadelphia, Pa., 17th ed. (1985).

In certain embodiments, the formulations of the present invention are administered to the host by a pulmonary route. In some embodiments, the pulmonary route of administration is in an inhalation dosage form directly into the respiratory tract, or directly to the respiratory airway, trachea, bronchi, bronchioles, lungs, alveolar ducts, alveolar sacs, and/or alveoli. The formulations may be administered by any convenient method, such as but not limited to: metered dose, nebulizers, atomizers, breath activated or powder. The methods of the present invention also include administrating the formulations directly into the nasal cavity or oral cavity of the host with a dropper, pipette or kanule.

In certain embodiments, the formulation is in a powder form. The agents may be used as a powder with a particle size ranging from about 1 to about 10 μm, such as from about 2 to about 8 μm. For pharmaceutical purposes the particle size of the powder may be no greater than about 100 μm diameter. In certain embodiments, the particle size of the finely-divided solid powder is about 25 μm or less, such as about 10 μm or less in diameter. The particle size of the powder for inhalation therapy may range from about 2 to about 10 μm.

The concentration of medicament depends upon the desired dosage. The precise therapeutic dosage amount will depend on the age, size, sex and condition of the subject, the nature and severity of the disorder, and other such factors. An ordinarily skilled physician or clinician can readily determine and prescribe the effective amount of the drug required for a particular patient.

In some embodiments, the formulations are powdered aerosol formulations which include the active agents suspended or dispersed in a propellant or a propellant and solvent. The propellant generally comprises a mixture of liquefied chlorofluorocarbons (CFCs) which are selected to provide the desired vapor pressure and stability of the formulation. Propellants 11, 12 and 114 are the most widely used propellants in aerosol formulations for inhalation administration. Other commonly used propellants include Propellants 113, 142b, 152a, 124, and dimethyl ether, which are commercially available from DuPont FluoroChemicals (Wilmington, Del.). The compound 1,1,1,2-tetrafluoroethane is also a commonly used propellant for medicinal aerosol formulations. The propellant comprises 40 to 90% by weight of the total inhalation composition.

The inhalation composition may also contain dispersing agents and solvents, such as phosphate buffer solution (PBS). Surfactants have also been used as dispersing agents. The surface active agents are generally present in amounts not exceeding 5% by weight of the total formulation. They may be present in the weight ratio 1:100 to 10:1 surface active agent to bisphosphonate active agent, but the surface active agent may exceed this weight ratio in cases where the drug concentration in the formulation is very low.

The inhalation formulation of the present invention can be delivered in any convenient inhalation device, where the device may include a nebulizer or an atomizer.

In the methods and compositions of the present invention, the pharmaceutical composition may be administered in admixture with suitable pharmaceutical diluents, excipients or carriers. Moreover, when desired or necessary, suitable excipients, lubricants, disintegrating agents and coloring agents can also be incorporated into the mixture of active ingredient(s) and inert carrier materials.

In some embodiments, the pharmaceutical composition is a powder formulation comprising a bisphosphonate active agent, or pharmacologically acceptable salt thereof, and one or more mucosal membrane protecting agents, e.g., SOD, taurine, cysteine and glutathione. In certain embodiments, the pharmaceutical composition further comprises one or more excipients, such as a plasticizer, lubricant, binder, disintegrator, stabilizer, or masking agent. In certain embodiments, the surface of the particles of the powder formulation are coated with a suitable coating agent. In certain embodiments, the pharmaceutical composition further comprises a lubricant, such as isopropyl myristate, light mineral oil or other substances which provide slippage between particles of the compound as well as lubrication for component parts of the valve of the inhalation device.

In some embodiments, the pharmaceutical composition is a solution or suspension formulation comprising a bisphosphonate active agent, or pharmacologically acceptable salt thereof, and one or more mucosal membrane protecting agents, e.g., SOD, taurine, cysteine and glutathione. In certain embodiments, the solution or suspension formulation comprises the agents dissolved or suspended in water. In certain embodiments, the solution or suspension formulation further comprises one or more co-solvents, such as, ethanol, propylene glycol, or polyethylene glycol. In certain embodiments, the solution or suspension formulation further comprises one or more preservatives, solubilizers, buffering agents, isotonizers, surfactants, absorption enhancers, or viscosity enhancers. In certain embodiments, the pharmaceutical composition is a suspension formulation and further comprises a suspending agent.

### Utility

The subject methods find use in a variety of applications, where in certain applications the methods are methods of modulating at least one cellular function, such as inhibiting bone reabsorption. The subject methods find use in treating, reducing the probability of, or preventing bone adsorption, loss of bone mass, osteoporosis, osteopenia, urolithiasis, hypercalcemia, Paget's disease (or osteitis deformans), bone metastasis, multiple myeloma, neoplastic bone lesions, and other conditions that cause or increase the risk of bone fragility. In some embodiments of the invention, the subject methods are also useful for reducing the probability or risk of non-vertebral fractures. In certain embodiments, the subject in need of the bisphosphonate active agent is osteoroporotic or postmenopausal, or both. In certain embodiments, the subject is a woman who is osteoroporotic or postmenopausal, or both. In certain embodiments, the subject is a human juvenile with osteogenesis imperfecta.

In this respect, the subject methods and composition find use in known applications of bisphosphonate, such as in treating diseases or disorders that are capable of being treated using bisphosphonate. Use of the subject compositions of the present invention is of particular utility in, for example, the treatment of diseases and disorders including but not limited to osteoporosis, osteopenia, urolithiasis, hypercalcemia, Paget's disease (or osteitis deformans), bone metastasis, multiple myeloma, neoplastic bone lesions, and other conditions that cause or increase the risk of bone fragility. In these capacities, use of the present inventive compositions will result in a reduced unwanted toxicity while retaining desired bisphosphonate activity.

As such, the subject methods and compositions find use in therapeutic applications in which bisphosphonate administration is indicated. A representative therapeutic application is the treatment of bone disease conditions, e.g., osteoporosis and related conditions characterized by bone adsorption and loss of bone mass.

By treatment is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans. In some embodiments, the hosts are women.

The subject methods find use in, among other applications, the treatment of bone disease conditions, including osteoporosis conditions. In such applications, an effective amount of the bisphosphonate active agent and mucosal membrane protecting agent(s) is administered to the subject in need thereof. Treatment is used broadly as defined above, e.g., to include at least an amelioration in one or more of the symptoms of the disease, as well as a complete cessation thereof, as well as a reversal and/or complete removal of the disease condition, e.g., cure.

The dose administered to an animal, particularly a human, in the context of the present invention should be sufficient to effect a prophylactic or therapeutic response in the animal over a reasonable time frame. One skilled in the art will recognize that dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound. Suitable doses and dosage regimens can be determined by comparisons to agents that are known to inhibit bone adsorption, particularly unmodified bisphosphonate. A suitable dosage is an amount which results in the inhibition of bone adsorption, without significant side effects. In proper doses and with suitable administration of certain compounds, the present invention provides for a wide range of intracellular effects, e.g., from partial inhibition to essentially complete inhibition of bone adsorption.

Individuals may be diagnosed as being in need of the subject methods using any convenient protocol, and are generally known to be in need of the subject methods, e.g., they are suffering from a target disease condition or have been determined to be at risk for suffering from a target disease condition, prior to practicing the subject methods.

Particular applications in which the subject methods and compositions find use include those described in U.S. Pat. Nos. 4,621,077; 5,183,815; 5,358,941; 5,462,932; 5,661,174; 5,681,590; 5,994,329; 6,015,801; 6,090,410; 6,225,294; 6,414,006; 6,482,411; and 6,743,414; the disclosures of which are herein incorporated by reference.

### Kits & Systems

Also provided are kits that find use in practicing the subject methods, as described above. For example, kits and systems for practicing the subject methods may include one or more pharmaceutical formulations, which include one or both of the bisphosphonate active agent and mucosal membrane protecting agent(s). As such, in certain embodiments the kits may include a single pharmaceutical composition, present as one or more unit dosages, where the composition includes both the bisphosphonate active agent and mucosal membrane protecting agent(s). In yet other embodiments, the kits may include two or more separate pharmaceutical compositions, each containing either a bisphosphonate active agent or a mucosal membrane protecting agent.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The term “system” as employed herein refers to a collection of bisphosphonate active agent(s) and mucosal membrane protecting agent(s) present in a single or disparate composition, that are brought together for the purpose of practicing the subject methods. For example, separately obtained bisphosphonate active agent(s) and mucosal membrane protecting agent(s) dosage forms brought together and coadministered to a subject, according to the present invention, are a system according to the present invention.

The following examples further illustrate the present invention and should not be construed as in any way limiting its scope.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### I. Administration Route Analysis

### A. Dosing Solution

2.5 mg/ml of Alendronate (by Toronto Research Chemicals Inc.) for venous administration and 12.5 mg/ml of Alendronate (by Toronto Research Chemicals Inc.) for transpulmonary administration were prepared by using the isotonic phosphate buffer solution (PBS) with the pH of 7.4.

12.5 mg/ml of Pamidronate (by Toronto Research Chemicals Inc.) for venous administration and 12.5 mg/ml of Pamidronate (by Toronto Research Chemicals Inc.) for transpulmonary administration were prepared by using the isotonic phosphate buffer solution (PBS) with the pH of 7.4.

B. Dosing Solution Added with Mucosal Protective Agent

12.5 mg/ml of Alendronate and Pamidronate, 16.7 mg/ml (50000 U/ml) of SOD, 50 mg/ml of cysteine, 125 mg/ml of taurine 50 mg/ml of glutathione for transpulmonary administration were prepared by using the isotonic phosphate buffer solution (PBS) with the pH of 7.4.

### C. Transpulmonary Administration

A transpulmonary absorption test was conducted as reported below (the following method is based on the method disclosed by Enna S J, Schanker L S.: Absorption of saccharides and urea from the rat lung. Am. J. Physiol., 222, 409-414 (1972)).

A Wistar male rat weighing 250 to 300 g was used in the test. Under pentobarbital anesthesia, the center of the neck of the rat was cut open to expose the bronchial tract. A 2.5 cm long polyethylene tube (ID 1.5 mm, OD 2.3 cm) was inserted from the thyroid cartilage between the 4^{th} and 5^{th} bronchial cartilage rings to a 0.6 cm depth, and the open skin was then stitched up. A 100 μl microsyringe (Microliter, no. 710, Hamilton Co) was filled with 100 μl of the dosing solution. The rat was placed at 800. The tip of the microsyringe was inserted at 1 to 2 mm up into the bronchial tract through the above polyethylene tube and the solution was administered in sync with the breath of the rat in 1 to 2 seconds. 5 mg/kg of Alendronate and 5 mg/kg of Pamidronate were administered to the rat by a pulmonary route. 45 seconds after the administration, the rat was placed at 100 and 250 μl of blood was sampled from the jugular vein in a time-dependent manner. The blood sample was centrifuged (13000 rpm, 10 min) to obtain the plasma fraction and it was stored at −30° right before the analysis.

### D. Venous Administration

A Wistar male rat weighing 250 to 300 g was used in the test. 1 mg/kg of Alendronate and 5 mg/kg of Pamidronate were administered to the rat through the femur vein. The blood sample was centrifuged (13000 rpm, 10 min) to obtain the plasma fraction and it was stored at −30° right before the analysis.

### E. Analysis Conditions

The assay for Alendronate and Pamidronate were conducted in the following method in reference with the report by Wong et al., “Determination of Pamidronate in human whole blood and urine by reversed-phase HPLC with fluorescence detection,” Biomed. Chromatogy. (2004) 18: 98-101. 120 μl of the plasma fraction obtained from the rat was diluted with 500 μl of ultrapure water. 75 μl of trichloroacetic acid (TCA) was added to remove protein and the mixture was centrifuged (13000 rpm, 5 min). The supernatant was filtered with a filter (0.45 μm).

Calcium chloride and monobasic sodium phosphate were added to 600 μl of the filtered supernatant. Sodium hydroxide was added to adjust the pH to 12 to sediment. The mixture was centrifuged and the sediment was washed with 500 μl of ultrapure water. Hydrochloric acid was added to the sediment to dissolve and sodium hydroxide was added to obtain the precipitate. After centrifuging, it was washed with 500 μl of ultrapure water and the sediment was dissolved in 100 μl of 50 mM Na₂EDTA (pH 10). After adding 30 μl of a fluorescamine/acetonitrile solution (3 mg florescamine/ml acetonitrile), 100 μl of dichloromethane was added to stir vigorously and centrifuged (13,000 rpm, 5 min). The obtained supernatant was collected and 10 μl of it, as an injection volume, was measured with the fluorescent-reverse-phase HPLC under the following conditions.
Equipment Used Shimadzu LC—10A system
Column: COSMOSIL C18 (4.6×150 mm)
Mobile Phase: 95% 1 mM Na₂EDTA—methanol ((97:3) pH 6.5 by 1N NaOH), 5% methanol
Flow Speed: 1.0 ml/min
Detector: Fluorescence detector (Ex: 395 nm, Em: 480 nm)
Column Temp.: 40°

### F. Results

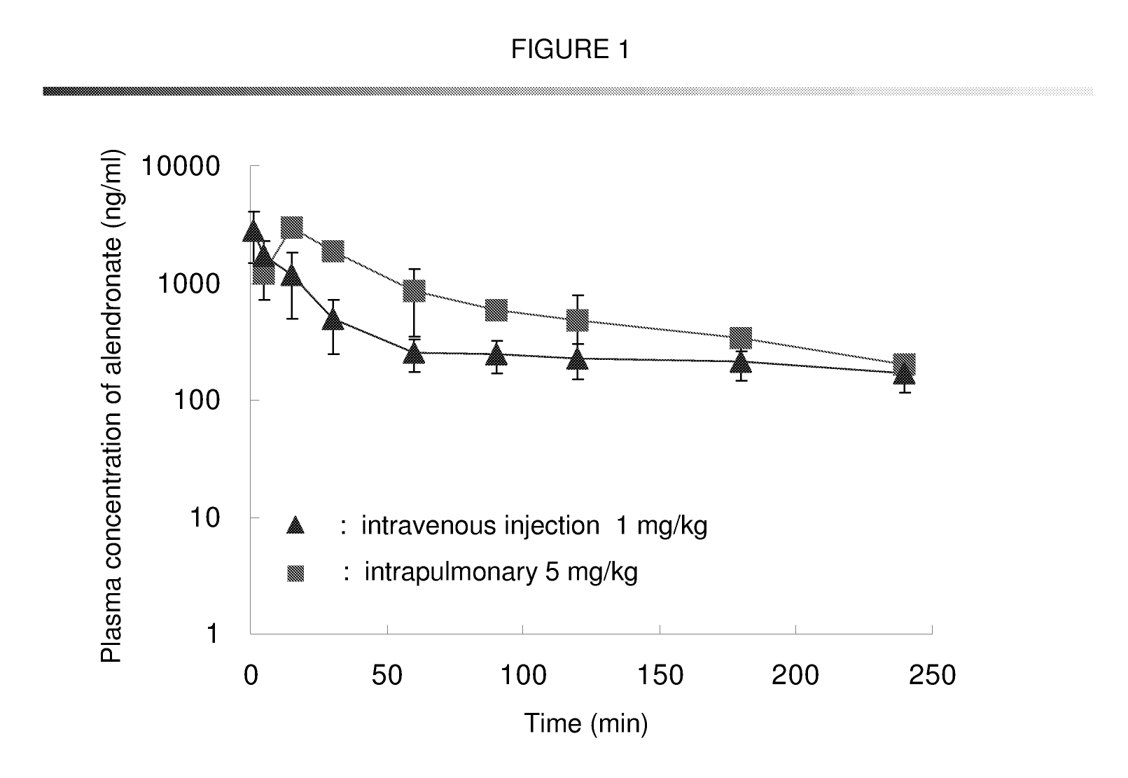

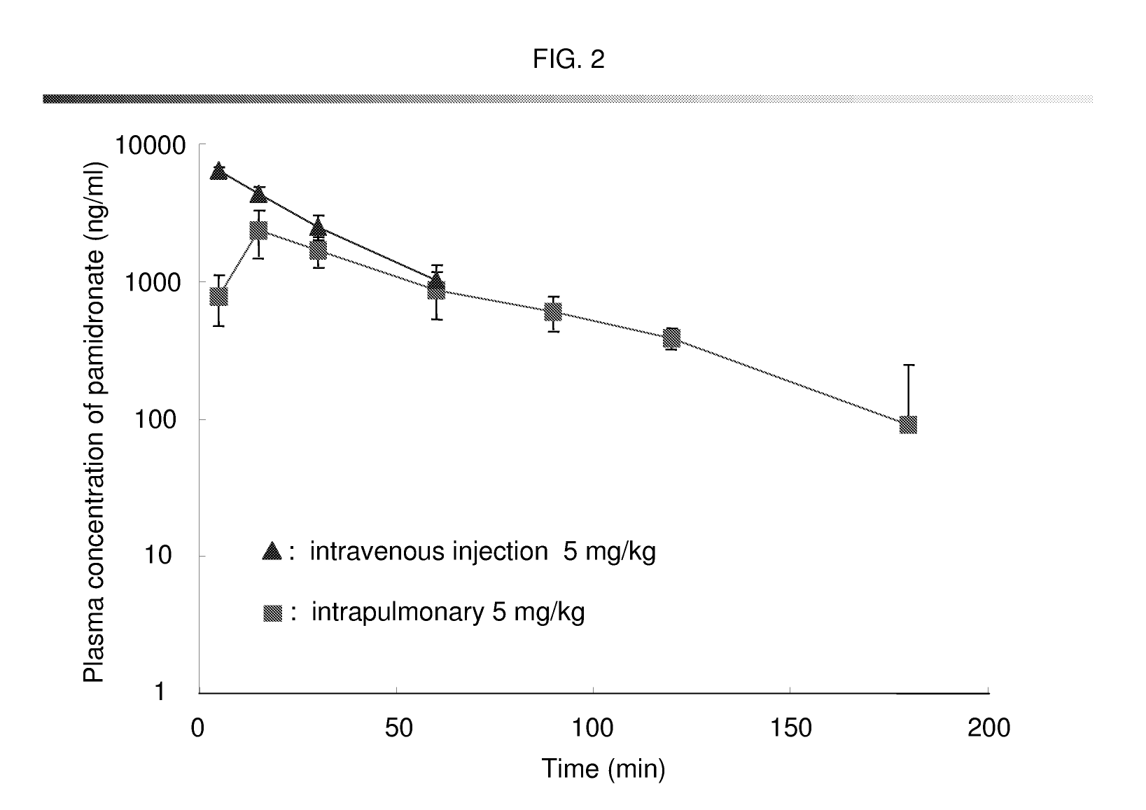

Results from the above analysis are shown in FIGS. 1 and 2.

### II. Pulmonary Inflammation Test.

This test is to measure the degree of irritation caused by a drug to a subject's pulmonary tract following administration of the drug by the pulmonary route. Following the liquid formulation administration, blood is removed from the aorta of the rat, and saline is injected from the pulmonary artery to wash the rat's lung with perfusion. The center of the neck is cut open to expose the bronchial tract, and a polyethylene tube is inserted to the bronchial tract to wash the bronchial tract with 16 mL of phosphate buffered saline (PBS) (4 washes of 4 mL each) (bronchialveolar lavage (BAL)). The derived BAL fluid (BALF) is centrifuged at 4° C., 200×g for 7 minutes, and the supernatant is sampled to measure the lactate dehydrogenase (LDH) activity.

LDH activity is assayed using the LDH-Cytotoxic Test (Wako Pure Chemical Industries, Ltd., Osaka, Japan). LDH is a stable enzyme which is present in all cell types. When the plasma membrane of a cell is damaged, LDH is rapidly released from the cell. Measuring the level of LDH activity in the serum is the most widely used marker in cytotoxicity studies. A high level of LDH activity detected indicates a high degree of irritation, while a low level of LDH activity detected indicates a low degree of irritation.

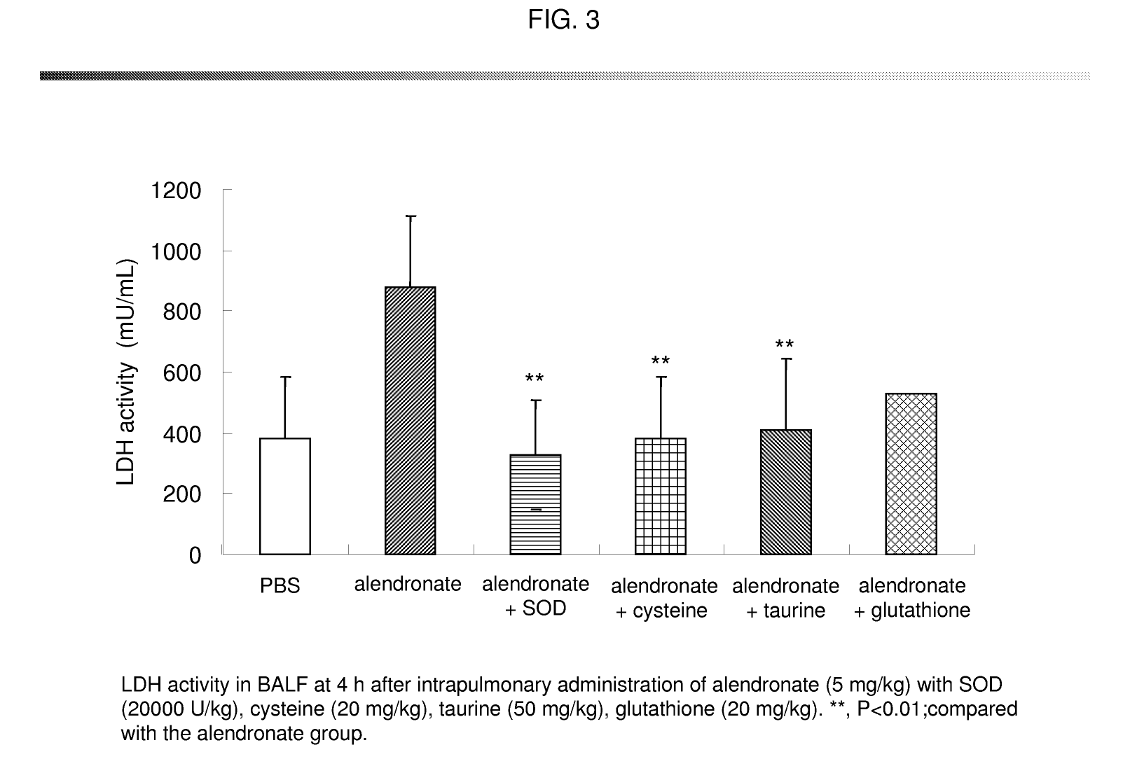

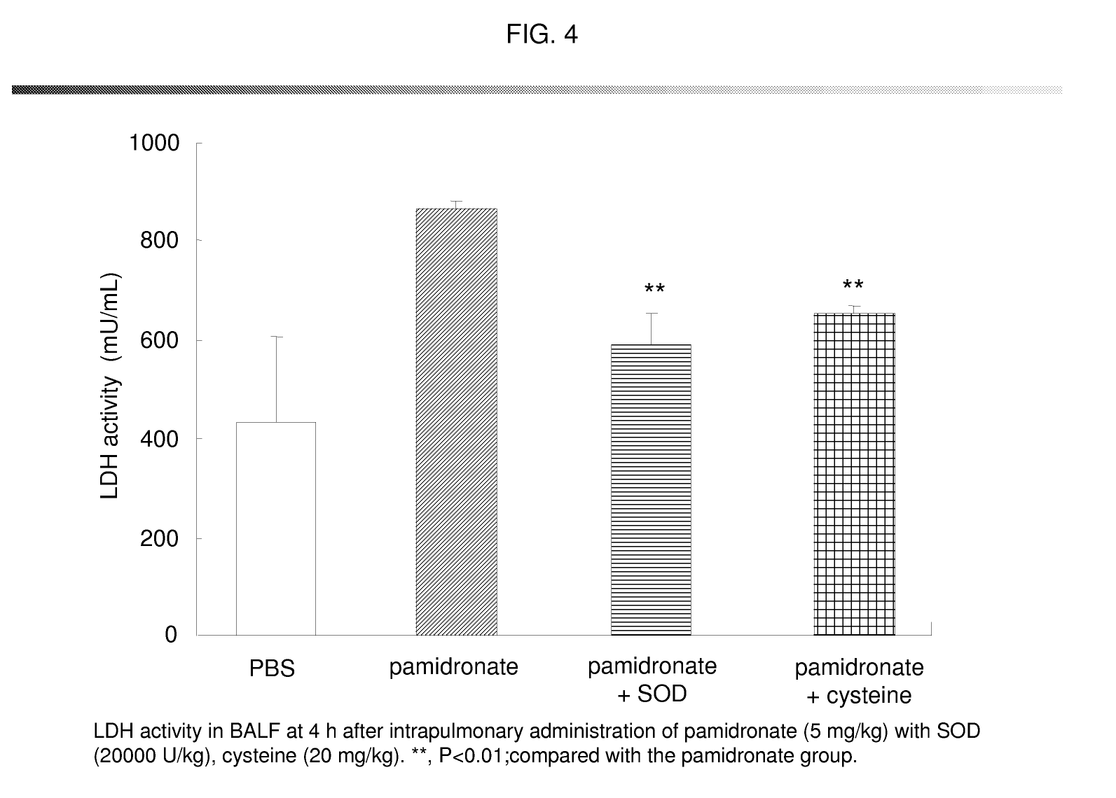

The results of this assay are provided in FIGS. 3 and 4.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of administering to a subject in need thereof an effective amount of a bisphosphonate active agent, said method comprising:
administering by a pulmonary route to said subject an effective amount of a bisphosphonate active agent in combination with a mucosal membrane protecting agent.

2. The method according to claim 1, wherein said bisphosphonate active agent is a compound of formula (I): 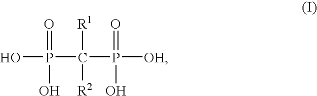
or the pharmaceutically acceptable salts, solvates, hydrates, and prodrug forms thereof, and stereoisomers thereof;
wherein:
R¹ is selected from the group consisting of hydrogen, —OH, and halogen; and
R² is selected from the group consisting of halogen, a linear or branched substituted or unsubstituted C₁-C₁₀ alkyl, a linear or branched substituted or unsubstituted C₁-C₁₀ cycloalkyl, a linear or branched substituted or unsubstituted C₁-C₁₀ aryl, a linear or branched substituted or unsubstituted C₁-C₁₀ aralkyl, a substituted or unsubstituted C₁-C₁₀ heterocycloalkyl, or a substituted or unsubstituted C₁-C₁₀ heteroaryl, wherein the each carbon atom of R² may be optionally replaced with a nitrogen or sulfur atom and R² has no more than 3 nitrogen or sulfur atoms in total.

3. The method according to claim 2, wherein said compound is a compound listed in Table 1.

4. The method according to claim 3, wherein said compound is alendronate.

5. The method according to claim 4, wherein said compound is a sodium salt of alendronate.

6. The method according to claim 3, wherein said compound is pamidronate.

7. The method according to claim 6, wherein said compound is a sodium salt of pamidronate.

8. The method according to claim 1, wherein said bisphosphonate active agent and said mucosal membrane protecting agent are administered simultaneously to said subject.

9. The method according to claim 8, wherein said bisphosphonate active agent and said mucosal membrane protecting agent are administered to said subject as separate formulations.

10. The method according to claim 8, wherein said bisphosphonate active agent and said mucosal membrane protecting agent are administered to said subject in a single formulation.

11. The method according to claim 1, wherein said bisphosphonate active agent and said mucosal membrane protecting agent are administered to said subject sequentially.

12. The method according to claim 11, wherein said bisphosphonate active agent is administered to said subject prior to said mucosal membrane protecting agent.

13. The method according to claim 11, wherein said bisphosphonate active agent is administered to said subject after said mucosal membrane protecting agent.

14. The method according to claim 1, wherein said mucosal membrane protecting agent is a protecting enzyme.

15. The method according to claim 14, wherein said protecting enzyme is selected from the group consisting of: superoxide dismutase (SOD), glutathione-S-transferase, glutathione reductase, catalase, and enzymatically active portions and variants thereof, and pharmaceutically acceptable salts, solvates, hydrates, and prodrug forms thereof, and stereoisomers thereof.

16. The method according to claim 15, wherein said protecting enzyme is SOD.

17. The method according to claim 1, wherein said mucosal membrane protecting agent is a protecting amino acid.

18. The method according to claim 17, wherein said protecting amino acid is selected from the group consisting of taurine and cysteine, and pharmaceutically acceptable salts, solvates, and derivatives thereof.

19. The method according to claim 18, wherein said protecting amino acid is taurine.

20. The method according to claim 18, wherein said protecting amino acid is cysteine.

21. The method according to claim 1, wherein said mucosal membrane protecting agent is a protecting peptide.

22. The method according to claim 21, wherein said protecting peptide is glutathione.

23. The method according to claim 1, wherein said method comprises administering to said subject at least two of a protecting enzyme, a protecting amino acid and a protecting peptide.

24. The method according to claim 23, wherein said method comprises administering to said subject a protecting enzyme, a protecting amino acid and a protecting peptide.

25. The method according to claim 1, wherein said pulmonary route comprises inhalation.

26. The method according to claim 1, wherein said method is of treating said subject for a bone adsorption disease.

27. The method according to claim 26, wherein said subject has been diagnosed as suffering from said bone adsorption disease.

28. The method according to claim 26, wherein said subject has been diagnosed as being at risk for suffering from said bone adsorption disease.

29. The method according to claim 26, wherein said bone adsorption disease is osteoporosis, osteopenia, urolithiasis, hypercalcemia, Paget's disease, bone metastasis, multiple myeloma, or neoplastic bone lesion.

30. A pharmaceutical composition comprising an effective amount of both a bisphosphonate active agent and a mucosal membrane protecting agent in a pharmaceutically acceptable vehicle.

31. The pharmaceutical composition according to claim 30, wherein said bisphosphonate active agent is a compound of formula (I): 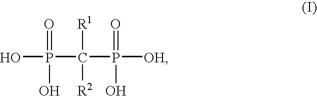
or the pharmaceutically acceptable salts, solvates, hydrates, and prodrug forms thereof, and stereoisomers thereof;
wherein:
R¹ is selected from the group consisting of hydrogen, —OH, and halogen; and
R² is selected from the group consisting of halogen, a linear or branched substituted or unsubstituted C₁-C₁₀ alkyl, a linear or branched substituted or unsubstituted C₁-C₁₀ cycloalkyl, a linear or branched substituted or unsubstituted C₁-C₁₀ aryl, a linear or branched substituted or unsubstituted C₁-C₁₀ aralkyl, a substituted or unsubstituted C₁-C₁₀ heterocycloalkyl, or a substituted or unsubstituted C₁-C₁₀ heteroaryl, wherein the each carbon atom of R² may be optionally replaced with a nitrogen or sulfur atom and R² has no more than 3 nitrogen or sulfur atoms in total.

32. The pharmaceutical composition according to claim 30, wherein said compound is a compound listed in Table 1.

33. The pharmaceutical composition according to claim 32, wherein said compound is alendronate.

34. The pharmaceutical composition according to claim 33, wherein said compound is a sodium salt of alendronate.

35. The pharmaceutical composition according to claim 32, wherein said compound is pamidronate.

36. The pharmaceutical composition according to claim 35, wherein said compound is a sodium salt of pamidronate.

37. The pharmaceutical composition according to claim 30, wherein said mucosal membrane protecting agent is a protecting enzyme.

38. The pharmaceutical composition according to claim 37, wherein said protecting enzyme is selected from the group consisting of superoxide dismutase (SOD), glutathione-S-transferase, glutathione reductase, catalase, and enzymatically active portions and variants thereof, and the pharmaceutically acceptable salts, solvates, hydrates, and prodrug forms thereof, and stereoisomers thereof.

39. The pharmaceutical composition according to claim 38, wherein the protecting enzyme is SOD.

40. The pharmaceutical composition according to claim 30, wherein said mucosal membrane protecting agent is a protecting amino acid.

41. The pharmaceutical composition according to claim 40, wherein said protecting amino acid is one selected from the group consisting of taurine and cysteine, and pharmaceutically acceptable salts, solvates, and derivatives thereof.

42. The pharmaceutical composition according to claim 41, wherein said protecting amino acid is taurine.

43. The pharmaceutical composition according to claim 41, wherein said protecting amino acid is cysteine.

44. The pharmaceutical composition according to claim 30, wherein said mucosal membrane protecting agent is a protecting peptide.

45. The pharmaceutical composition according to claim 44, wherein said protecting peptide is glutathione.

46. The pharmaceutical composition according to claim 30, wherein said composition comprises two or more of a protecting enzyme, a protecting amino acid and a protecting peptide.

47. The pharmaceutical composition according to claim 46, wherein said protecting enzyme is SOD, said protecting amino acid is taurine or cysteine and said protecting peptide is glutathione.

48. The pharmaceutical composition according to claim 30, wherein said composition comprises a protecting enzyme, a protecting amino acid and a protecting peptide.

49. The pharmaceutical composition according to claim 30, wherein said pharmaceutical composition is an aerosol.

50. The pharmaceutical composition according to claim 49, wherein said aerosol is a liquid aerosol.

51. The pharmaceutical composition according to claim 49, wherein said aerosol is a solid aerosol.

52. The pharmaceutical composition according to claim 51, wherein said solid aerosol comprises a dry powder.

53. The pharmaceutical composition according to claim 52, wherein said powder comprises particles ranging in size from about 1 to about 100 μm.

54. A kit for use in treating a subject suffering from a bone adsorption disease condition, said kit comprising:
(a) a bisphosphonate active agent; and
(b) a mucosal membrane protecting agent.

55. The kit according to claim 54, wherein said mucosal membrane protecting agent is a protecting enzyme.

56. The kit according to claim 54, wherein said mucosal membrane protecting agent is a protecting amino acid.

57. The kit according to claim 54, wherein said mucosal membrane protecting agent is a protecting peptide.

58. The kit according to claim 54, wherein said kit comprises at least two of a protecting enzyme, a protecting amino acid and a protecting peptide.

59. The kit according to claim 58, wherein said protecting enzyme is SOD, said protecting amino acid is taurine or cysteine, and said protecting peptide is glutathione.
